(19) United States
(12) Patent Application Publication
(10) Pub. No.: US 2008/0004673 A1
(43) Pub. Date: Jan. 3, 2008

(54) IMPLANTABLE EXTRAVASCULAR ELECTROSTIMULATION SYSTEM HAVING A RESILIENT CUFF

(75) Inventors: Martin A. Rossing, Coon Rapids, MN (US); Mary L. Cole, St. Paul, MN (US); Brian Soltis, St. Paul, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Maple Grove, MN (US)

(21) Appl. No.: 11/695,210

(22) Filed: Apr. 2, 2007

Related U.S. Application Data
(60) Provisional application No. 60/789,208, filed on Apr. 3, 2006.

(51) Int. Cl. A61N 1/05

(52) U.S. Cl. 607/44

(57) ABSTRACT

A method and device for providing stimulation to an artery for purposes of eliciting a physiologic response. A cuff having at least one electrode is provided, wherein the cuff is biased to conform to at least a portion of a vascular structure to maintain an intimate vascular structure-electrode interface. The device is selectively positioned proximate the effective position for providing stimulation to the vascular structure and the cuff is enabled to biasedly conform to at least a portion of the vascular structure. The cuff comprises includes resiliency enabling substantially normal pulsatile expansion of the artery while maintaining effective artery-electrode interface.

### CROSS-REFERENCES TO RELATED APPLICATIONS

The present application claims the benefit of U.S. Provisional Application No. 60/789,208 filed Apr. 3, 2006, which is incorporated herein in its entirety by reference.

### BACKGROUND OF THE INVENTION

### Field of the Invention

This invention relates generally to implantable medical devices. More particularly, the present invention relates to methods and apparatus for providing an extravascular electrode assembly having a resilient cuff as part of a baroreflex activation device to facilitate positioning the electrodes about a desired surface of a biological vessel structure such as an artery or a vein.

Cardiovascular disease is a major contributor to patient illness and mortality. It is also a primary driver of health care expenditure, costing more than $326 billion each year in the United States. Hypertension, or high blood pressure, is a major cardiovascular disorder that is estimated to affect over 50 million people in the United Sates alone. Of those with hypertension, it is reported that fewer than 30% have their blood pressure under control. Hypertension is a leading cause of heart failure and stroke. It is the primary cause of death in over 42,000 patients per year and is listed as a primary or contributing cause of death in over 200,000 patients per year in the U.S. Accordingly, hypertension is a serious health problem demanding significant research and development for the treatment thereof.

Hypertension occurs when the body's smaller blood vessels (arterioles) constrict, causing an increase in blood pressure. Because the blood vessels constrict, the heart must work harder to maintain blood flow at the higher pressures. Although the body may tolerate short periods of increased blood pressure, sustained hypertension may eventually result in damage to multiple body organs, including the kidneys, brain, eyes and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke. Sustained high blood pressure may eventually result in an enlarged and damaged heart (hypertrophy), which may lead to heart failure.

It has been known for decades that the wall of the carotid sinus, a structure at the bifurcation of the common carotid arteries in the neck, contains stretch receptors known as baroreceptors that are sensitive to blood pressure. These receptors send signals via the carotid sinus nerve to the brain, which in turn regulates the cardiovascular system to maintain normal blood pressure (the baroreflex), in part through activation of the sympathetic nervous system. Electrical stimulation of the carotid sinus nerve (baropacing) has previously been proposed to reduce blood pressure and the workload of the heart in the treatment of high blood pressure and angina. For example, U.S. Pat. No. 6,073,048 to Kieval et al. discloses a baroreflex modulation system and method for stimulating the baroreflex that are based on various cardiovascular and pulmonary parameters.

Implantable electrode assemblies for electrotherapy or electrostimulation of vessels in the body are known in the art. For example, various configurations of implantable electrodes are described in U.S. Patent Publication No. U.S. 2004/0010303, which is incorporated herein by reference in its entirety. One type of vessel electrode exterior assembly described therein is an exterior surface-type stimulation electrode that generally includes a set of generally parallel elongate electrodes secured to, or formed on, a common substrate or base. Prior to implantation in a patient, the electrodes in the electrode assembly are generally electrically isolated from one another. Once the exterior vessel electrode assembly is implanted about the desired vessel, it is secured in location, such as by suturing, and one or more of the electrodes are utilized as a cathode(s), while one or more of the remaining electrodes are utilized as an anode(s). The implanted cathode(s) and anode(s) are thus electrically coupled via the target region of tissue to be treated or stimulated.

The process of implanting an exterior vessel electrode assembly for baroreceptor stimulation involves positioning the assembly such that the electrodes are properly situated against the arterial wall of the carotid sinus, and securing the vessel electrode assembly to the artery so that the positioning is maintained. The positioning is a critical step because the electrodes must direct as much energy as possible to the baroreceptors for maximum effectiveness and efficiency. The energy source for the implanted baroreflex stimulation device is typically an on-board battery with finite capacity. A high-efficiency implantation of the exterior vessel electrodes will provide a longer battery life and correspondingly longer effective service life between surgeries because less energy will be required to achieve the needed degree of therapy. As such, during implantation of the vessel electrode assembly, the position of the assembly is typically adjusted several times in order to optimize the baroreflex response.

This process of adjusting and re-adjusting the position of the electrode assembly, known as mapping, has been reported by surgeons as difficult and tedious. Present-day procedures involve positioning and holding the exterior vessel electrode assembly in place with tweezers, hemostat or similar tool while applying the stimulus and observing the response in the patient. Movement by as little as 1 mm can make a difference in the effectiveness of the baroreflex stimulation.

Another challenge related to the mapping process is keeping track of previous desirable positions. Because mapping is an optimization procedure, surgeons will tend to search for better positions until they have exhausted all reasonable alternative positions. Returning the electrode assembly to a previously-observed optimal position can be quite difficult and frustrating, especially under the surgical conditions.

After determining the optimal position, the surgeon must secure the electrode assembly in place. In the system described, for example, in U.S. Patent Application No. 2004/0010303 A1, entitled “Electrode Structures and Methods for their Use in Cardiovascular Reflex Control” this has been accomplished by wrapping finger-like elongated portions of the electrode assembly around the artery, applying tension to the material, and suturing the assembly in place. The electrode assembly can be sutured to the arterial wall or to itself (after being wrapped around the artery). Loosening or removing the sutures, re-positioning the electrode assembly, and tightening or re-installing the sutures can increase the time and costs associated with implanting such baropacing devices, and can also increase the risk of complications or surgeon errors related to protracted surgical procedures and fatigue.

Medical devices employing a cuff adapted to engage with a biological structure have been used to treat various conditions. For example, U.S. Pat. No. 4,602,624, entitled “Implantable Cuff, Method Of Manufacture, and Method Of Installation,” relates to a cuff having a self-curling sheet of non-conductive material which is self-biased to curl into a tight spiral or roll. U.S. Pat. No. 4,602,624 entitled “Implantable Cuff, Method of Manufacture, and Method of Installation” discloses that the cuffs can be disposed around nerve trunks in order to provide electrical stimulation of the nervous system. U.S. Pat. No. 5,038,781, entitled “Multi-Electrode Neurological Stimulation Apparatus,” discloses a nerve cuff having the general shape of a gapped hollow cylinder that can be applied to a nerve. U.S. Published Application No. 2003/0216792, entitled “Renal Nerve Stimulation Method And Apparatus For Treatment Of Patients,” discloses a cuff that can envelope a renal artery in order to stimulate the renal nerve. However, none of the above references disclose devices or methods specifically adapted to engage with the carotid sinus artery or for use as part of a baroreflex activation therapy system.

Accordingly, there continues to be a substantial need for new electrode devices and methods for treating and/or managing high blood pressure, heart failure and their associated cardiovascular and nervous system disorders.

### BRIEF SUMMARY OF THE INVENTION

The present invention is directed to an implantable extravascular system for applying electrostimulation that may comprise a cuff having body portion and at least one electrode operably coupled to the body portion and adapted to contact an exterior tissue surface when the cuff is engaged with a biological vascular structure such as a vein or artery. The body portion can be formed from a resilient material such as, for example, silicone rubber and can be adapted to engaged and disengage with desired biological structures without the use of sutures or other fastening elements, which can make the mapping or positioning process of the cuff less difficult and time consuming. More specifically, the body portion of the cuffs is selectively or otherwise selectively shiftable between an open position that allows placement of the cuff about a biological structure and a closed position that generally retains the cuff in a desired position along the biological structure. In some embodiments, the body portion of the cuff can be biased towards the closed position while still permitting the substantially unimpeded natural operation of the baroreceptors in the vessel wall of the vascular structure.

In some embodiments, the body portion of the implantable vessel electrode can be formed from a single resilient material such as, for example, silicone rubber; while in other embodiments the body portion can comprise a composite formed from two or more resilient materials. For example, the body portion can comprise a memory metal encapsulated within a suitable polymeric and/or elastomeric material. In one embodiment, the cuff can be designed to impart a suitable biasing force such that the body portion is biased towards a closed position, which facilitates operably coupling the cuff to a desired biological structure without the use of sutures or other mechanical fasteners and with minimal reduction in the nominal diameter of the vessel that would cause a potential reduction of blood flow through the vessel. In some embodiments, a vessel such as an artery may expand up to 10% or more in diameter in response to pulse pressure. For example, a natural radial expansion of up to 6% of an artery can be observed with a pulse pressure of approximately 40-50 mmHg. Under such conditions, the biasing force would be sufficient to permit the cuff to remain operationally intact with the artery and while limiting the expansion of the artery by less than 50% from its natural expansion. In one embodiment, the natural radial expansion of an artery can be permitted up to 4% with the cuff of the present invention in position about the artery.

The body portion of the cuff can comprise a hollow generally cylindrical body portion, wherein the body portion defines a gap or opening that permits access into the hollow interior of the body portion. In these embodiments, the body portion can be shifted or deformed with respect to a longitudinal axis to allow adjustment of the gap from a closed position to an open position for placement of the cuff around a biological vessel structure such as, for example, an artery or a vein. In other embodiments, the cuff can include a body portion comprising a self-curling sheet that can be shifted from a closed curled position to an open position for placement of the cuff around a biological structure. In some embodiments, the self-curling sheet can be biased towards the closed curled position.

In one aspect, the invention pertains to a method of activating a baroreceptor to induce a desired baroreceptor signal comprising the step of positioning a cuff about an artery in the region of the carotid sinus. In these embodiments, the cuff can comprise a body portion and an electrode assembly on a surface of the body portion adapted to contact an exterior surface of the artery when the cuff is engaged with the artery, wherein the body portion is formed from a resilient material and is shiftable from a closed position to an open position for selective placement of the cuff about the artery.

In another aspect, the invention pertains to an implantable extravascular electrostimulation device comprising a cuff having body portion and an electrode assembly on a surface of the body portion, wherein the body portion is formed from a resilient material and is selectively shiftable from a closed position to an open position for placement of the cuff about a carotid sinus artery, wherein the body portion is biased towards the closed position such that the cuff remains in contact with the carotid sinus artery while normal pulsatile expansion is reduced between about 0% and about 80%.

Additional ranges within the explicit range of about 0% to about 80% are contemplated and are within the present disclosure. Specifically, cuff can comprise resiliency sufficient to keep said electrode structure in contact with the artery without impeding pulsatile expansion by more than about 80% percent with a pulse pressure of up to about 50 mm Hg. In an embodiment, resiliency is sufficient to keep said electrode structure in contact with the artery without impeding pulsatile expansion by more than about 60% percent with a pulse pressure of up to about 50 mm Hg. In a further embodiment, resiliency is sufficient to keep said electrode structure in contact with the artery without impeding pulsatile expansion by more than about 40% percent with a pulse pressure of up to about 50 mm Hg. In yet a further embodiment, resiliency is sufficient to keep said electrode structure in contact with the artery without impeding pulsatile expansion by more than about 40% percent with a pulse pressure of up to about 50 mm Hg.

In a further aspect, the invention pertains to an implantable exterior vessel electrostimulation device comprising a cuff having a body portion with three electrodes on a surface of the body portion adapted to contact an exterior of the vessel structure when the cuff is engaged with the biological structure, wherein the body portion comprises a generally C-shaped cross section and defines a gap that can be shifted from an open position to a closed position, wherein the body portion is biased towards the closed position and wherein the gap in the open position is generally larger than the biological vessel structure to which the cuff is to be applied.

### BRIEF DESCRIPTION OF THE DRAWINGS

FIG. 1 is a front perspective view of an embodiment of a resilient cuff having a body portion comprising a self-curling sheet

FIG. 2 is a back perspective view of the cuff of FIG. 1.

FIG. 3 is a perspective view of the cuff of FIG. 1 being positioned around a carotid sinus artery.

FIG. 4 is a top view of a resilient cuff having fingers that can engage with buckle structures to additionally secure the cuff to a biological structure such as an artery or a vein.

FIG. 5 is a top view of the resilient cuff of FIG. 4, wherein the cuff is positioned around the carotid sinus artery.

FIG. 6 is a top view of a finger portion having a triangular pattern of suture site located on a surface of the finger portion.

FIG. 7 is a perspective view of an embodiment of a resilient cuff having a generally cylindrical body portion positioned around a carotid sinus artery, wherein the body portion defines a gap or opening that permits access into the hollow interior of the body portion.

FIG. 8 is a front perspective view of an embodiment of a resilient cuff, wherein the resilient cuff is separate from a first cuff including an electrode structure.

FIG. 9 is a front perspective view of a further embodiment of a resilient cuff, wherein the resilient cuff is separate from a first cuff including an electrode structure.

While the invention is amenable to various modifications and alternative forms, specific examples shown in the drawings will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

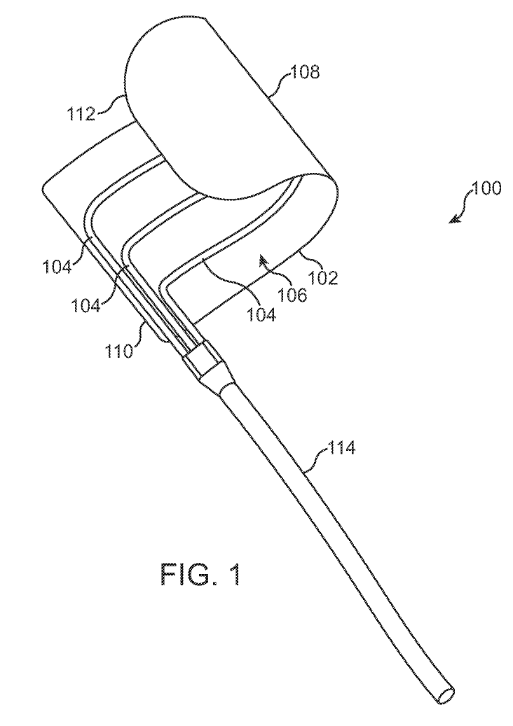

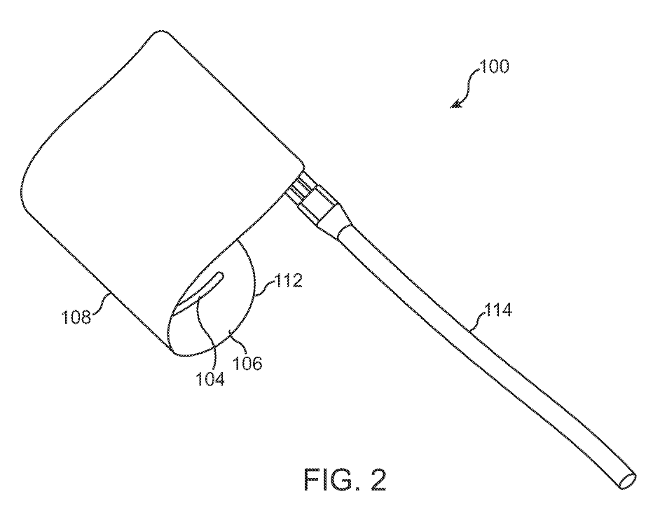

Referring to FIGS. 1-3, an implantable exterior vessel electrostimulation system 100 is depicted comprising a resilient cuff having a body portion 102 and an electrode assembly having a plurality of electrodes 104 positioned on a surface of body portion 102. As depicted in FIGS. 1-3, body portion 102 can be a self curling sheet having a first generally planar surface 106 and second generally planar surface 108 opposite first surface 106. In some embodiments, the electrode assembly can comprise three electrodes 104 positioned on first surface 106, although embodiments exist where medical device 100 comprises, for example, 2 and 4-6 electrodes positioned on first surface 106. One of ordinary skill in the art will recognize that the number of electrodes employed in a particular system can be guided by the intended application of the device. The self curling sheet can extend from a first edge 110 to a second edge 112. In some embodiments, electrodes 104 can extend substantially across first surface 106 of body portion 102 from second edge 112 to first edge 110 and into sheath 114, which is positioned proximate first edge 110.

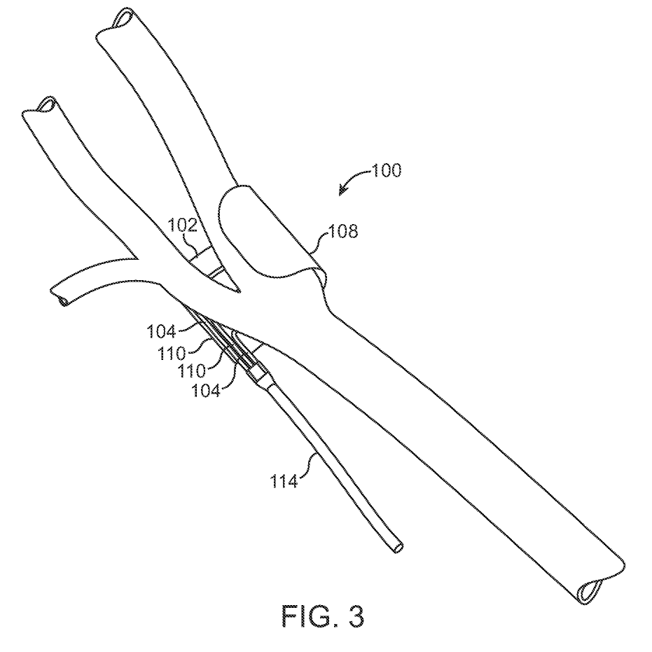

Generally, the self curling sheet is selectively shiftable from an open position to a closed position and is biased towards the closed position, which facilitates placement of body portion 102 around a desired biological vessel structure such as, for example, an artery, vein, or the like. In the open position, body portion 102 may be less curled or substantially flat, which allows placement first surface 106 of body portion 102 proximate a desired biological vessel structure. In the closed position, second edge 112 curls towards first surface 106, which wraps body portion 102 around a desired biological vessel structure and secures body portion 102 to a desired biological vessel structure. FIG. 3 depicts body portion 102 wrapping around a carotid sinus artery.

As described above, the self-curling sheet can be biased towards a closed or curled position. The biasing force is generally sufficient to acutely or chronically hold body portion 102 around a desired biological vessel structure such that body portion 102 does not disengaged from the biological vessel structure. Additionally, the biasing force preferably keeps body portion 102 curled tightly enough around the biological structure so that electrodes 104 remain in contact with desired exterior surfaces of the biological vessel structure but not so tight as to cause the body portion 102 to overly restrict blood flow in the biological vessel structure. For example, body portion 102 can be sized to fit around the carotid sinus artery and can have a sufficient biasing force to hold body portion 102, and electrodes 104, in contact with desired surfaces of the carotid sinus artery. In some embodiments, a vessel such as an artery may expand 6% with a pulse pressure of approximately 40-50 mmHg. Under such conditions, the biasing force would be sufficient to remain in contact with the artery and preferentially reduce the expansion of the artery by less than 4%.

First surface 106 can further include one or more additional chronic securing elements to further chronically securing body portion 102 to desired portions of a biological structure. Generally, the additional securing elements can be any element suitable to hold body portion 102 in contact with desired surfaces of a biological structure, or create additional frictional or locking engagement between surface 106 and a surface of a biological structure. Suitable additional securing elements include, for example, biological glue, adhesives strips, a plurality of protrusions extending from first surface 106, a hook and loop mechanism (e.g., similar to VELCRO® mechanism), textured or undulated surfaces, and combinations thereof. In one embodiment, the protrusions can comprise mushroom shaped protrusions that extend from first surface 106 to provide frictional engagement with surfaces of desired biological structures.

Care generally can be taken when acutely and/or chronically securing body portion 102 on a biological structure, such as near the baroreceptors at the carotid sinus. Specifically, as discussed, a vessel such as an artery may expand 6% with a pulse pressure of approximately 40-50 mmHg. Securement of the system on a vessel should not restrict such pulsatile expansion, as such restriction could affect baroreceptor functioning. Specifically, restriction of the expansion can act as a contraction on the artery. This can cause a false parameter indicative of the need to modify the baroreflex system activity causing the control system to generate a control signal activating the baroreceptor activation device to induce a baroreceptor signal that is perceived by the brain to be apparent excessive blood pressure. In a worst case scenario, the baroreceptors may become inactive due to a substantial lack of expansion. Thus, body portion 102 can have sufficient resiliency to enable expansion of the vessel while maintaining effective vessel-electrode contact.

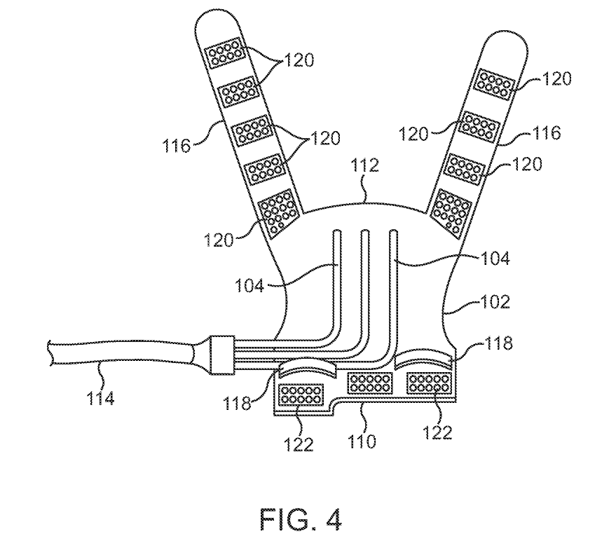

Chronic securing mechanisms such as those as listed above (e.g., sutures or biological glue) can be selectively presented on body portion 102, such as along first edge 110 thereof to provide such resiliency. For example, FIG. 4 depicts suture sites 122 along first edge 110. Chronic securing mechanisms could provide fixation to the biological vessel in which the electrode is attached, or it could provide fixation to a branch vessel. FIG. 3 depicts the common carotid, external carotid and internal carotid artery. In this figure, the securing mechanisms could be presented on the external carotid artery, common carotid artery or internal carotid artery even though the carotid sinus on the internal carotid artery is the intended target for stimulation. For brevity, examples such as this will be referred to as one vessel. In this configuration, the biasing force of body portion 102 and the chronic securing mechanism along first edge 110 can together chronically secure body portion 102 in contact with a desired surface of a biological structure. Because second edge 112 is not secured to the biological structure, pulsatile expansion is not overly inhibited or interfered with, thus not affecting baroreceptor functioning. In addition, the application of chronic securing mechanisms, such as sutures or biological glue, can enable ease of implantation, as portions of said cuff (e.g., second edge 112) can often be hidden, which could otherwise make a portion of the implementation procedure “blind.”

As another example of selectively chronic securing body portion 102, chronic securing mechanism (e.g., sutures or biological glue) can be presented at selective positions on body portion 102 away from the baroreceptors. In this configuration, the biasing force of body portion 102 and the chronic securing mechanism at points or positioned distal from the baroreceptors can function to chronically secure body portion 102 in contact with desired surfaces of a biological structure, while not overly inhibiting pulsatile expansion or interfere with baroreceptor functioning.

In yet a further embodiment, surface features, such as texturing or materials promoting tissue in-growth, can be included on surface 106. Such texturing or materials can enable tissue growth into surface 106, such that the tissue-surface 106 interface can act as a chronic securing mechanism. However, when texturing or other surface features are included on surface 106, care can be taken when placing an extravascular activation device near the baroreceptors at the carotid sinus, as any friction between the device and vascular wall can present potential for damage to the outer wall of a vascular lumen. The spatial pitch between electrodes 104 can enable more or less tissue-surface 106 interfacing for more or less chronic securement. For example, greater spatial pitch between electrodes enables more surface area of a vessel-surface 106 interfacing.

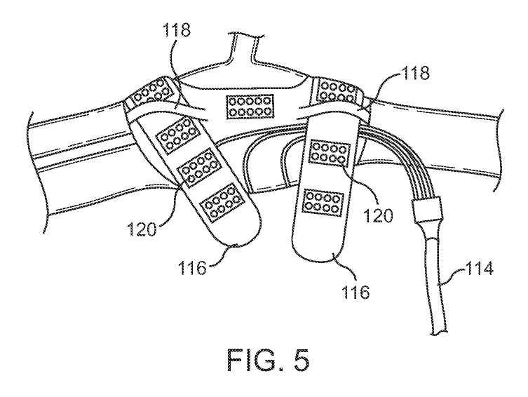

Referring to FIGS. 4 and 5, body portion 102 can further comprise one or more fingers 116 that extend from body portion 102. Fingers 116 can be adapted to wrap around a biological vessel structure and fit into, or engage with, buckles 118 formed onto body portion 102 to further facilitate securing body portion 102 to the biological structure. Generally, buckles 118 can be any structure adapted to receive and secure fingers 116 such as a slit or opening in the surface of body portion 102, a tab that can hold fingers 116 between the tab and body portion 102, a protrusion adapted to engage with a recess or opening formed into fingers 116, and combinations thereof. Buckles 118 can be provided to first surface 106 and/or second surface 108 of body portion 102. Buckle/strap and protrusion/recess configurations are described in greater detail in U.S. Patent Application No. 60/805,707, entitled “Implantable Electrode Assembly Utilizing a Belt Mechanism for Sutureless Attachment,” which is incorporated herein by reference in its entirety.

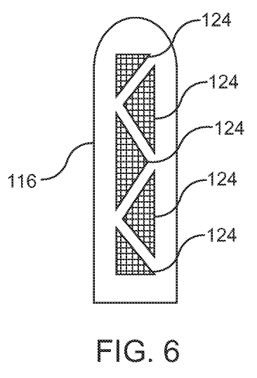

Additionally, fingers 116 can comprise one or more suture sites 120, which allow fingers 116 to be sutured, via corresponding suture sites 122, to body portion 102 to further secure the device around a desired biological vessel structure once the mapping process has been completed. In some embodiments, fingers 116 can be formed from an elastomer such as, for example, silicone rubber, and suture sites 120, 122 can be formed from a polyester fiber such as, for example, Dacron®. With respect to FIG. 6, in some embodiments, fingers 116 can have a plurality of triangular suture sites 124 arranged on a surface of the finger to minimize the distance between adjacent suture sites. The triangular shaped suture sites 124 allow for closer packing of the suture sites along a surface of finger 116, and thus provide more suture sites on a particular finger 116. As a result, the triangular suture sites make it easier to suture finger 116 to body portion 102 at desired locations along finger 116.

As described above, the resilient cuffs of the present invention comprise a body portion 102 and an electrode assembly positioned on a surface of body portion 102. The electrode assembly can include two or more elongate electrodes 104 for making contact with the target tissue region into which electrotherapy or electrostimulation is to be applied. As depicted in FIGS. 1-3, body portion 102 can include three electrodes 104, however, persons skilled in the relevant arts will recognize that electrode assemblies with at least two electrodes, and electrode assemblies with more than three electrodes are contemplated and are within the scope of the present disclosure. The electrodes can be un-insulated portions of larger electrical conductors, dedicated un-insulated conductive structures, or a combination thereof. In one example embodiment, elongate electrodes 104 are each about the same length, and are situated generally parallel to one another.

In a related type of embodiment, the electrodes are generally co-extensive. Among electrode assemblies of this type, the extent of co-extensiveness can vary according to the geometry of the implantation site. For example, in one example embodiment, the electrodes are co-extensive to within +/−25%. In another embodiment, the electrodes are co-extensive to within +/−5%. While this embodiment features one arrangement of three electrodes 104 in accordance with the present invention, other arrangements and configurations of electrodes 104 as described hereinafter may also be utilized to enhance the uniform distribution of the electric field delivered through the electrodes to the target tissue region. Various configurations of implantable electrodes are described in U.S. Patent Publication No. U.S. 2004/0010303, entitled “Electrode Structures And Methods For Their Use In Cardiovascular Reflex Control,” and in U.S. Patent Publication No. U.S. 2003/0060857, entitled “Electrode Designs And Methods Of Use For Cardiovascular Reflex Control Devices,” both of which are hereby incorporated by reference herein.

Electrodes 104 can be made from any suitable implantable material, and are preferably adapted to have flexible and/or elastic properties. Electrodes 104 can comprise round wire, rectangular ribbon or foil formed of an electrically conductive and radiopaque material such as platinum. In one embodiment, body portion 102 substantially encapsulates the conductive material, leaving only exposed electrode 104 portions for electrical connection to the target tissue. For example, each conductive structure can be partially recessed in body portion 102 and can have one side exposed along all or a portion of its length for electrical connection to target tissue. The exposed portions constitute electrodes 104.

In another embodiment, electrodes 104 can be made from conductive structures that can be adhesively attached to body portion 102 or can be physically connected by straps, moldings or other forms of operably securing them to the body portion 102. Electrical paths through the target tissue are defined by anode-cathode pairs of the elongate electrodes 104. For example, in one embodiment, the center electrode is a cathode, and the outer electrodes are both anodes, or vice-versa. Thus, electrons of the electrotherapy or electrostimulus signaling will flow through the target region either into, or out of, the center electrode.

Each of the plurality of electrodes 104 is connected at the corresponding proximal end to an electrotherapy/electrostimulus source, such as an implantable pulse generator (not shown) via a corresponding lead. In one example embodiment, the leads are each an insulated wire formed with, welded to, or suitably interconnected with each corresponding electrode 104. Persons skilled in the art will appreciate that the leads can be made of any suitable materials or geometries. Furthermore, the leads can each include a combination of conductor types. Thus, for example, the leads can each include an insulated stranded wire portion, an un-insulated solid wire portion, and/or a coiled wire portion having helical, spiral, or other such coiled geometry.

Body portion 102 can be formed from any material suitable for medical device applications including, for example, elastomers, polymers, memory metals, memory polymers, biodegradable polymers, and combinations thereof. In one embodiment, body portion 102 can be formed from a single material such as silicone rubber, while in other embodiments body portion 102 can be formed by encapsulating a memory metal such as Nitinol or other shape memory alloy in a suitable polymer and/or elastomer. Another embodiment could use a memory polymer such as an oligo dimethacrylate as a single material or in combination with other polymers. Yet another embodiment could use a biodegradable polymer such as polycaprolactone in combination with a non-biodegradable polymer to attain a more desirable closed position with the reduction of the biodegradable polymer. In other embodiments, a first layer can be operably coupled to a second layer to form body portion 102. In these embodiments, the first layer can comprise silicone rubber, while the second layer can comprise silicone rubber, a polytetrafluoroethylene (PTFE) film, a metal mesh such as a platinum mesh, or combinations thereof. Self-curling sheets formed from a first layer laminated to a second layer are described in U.S. Pat. No. 4,602,624, entitled “Implantable Cuff, Method Of Manufacture, And Method Of Installation,” which is hereby incorporated by reference herein.

In embodiments where body portion 102 is formed from a polymer and/or elastomer, the polymer and/or elastomer can comprise an additive which can be released from body portion 102 to provide site specific delivery of the additive. Suitable additives include, for example, antibiotics, other pharmaceutical agents, steroid elution materials, and combinations thereof. Generally, the additives are present in the polymer or elastomer at a concentration of less than about 5 percent by weight, and more preferably less than about 1 percent by weight.

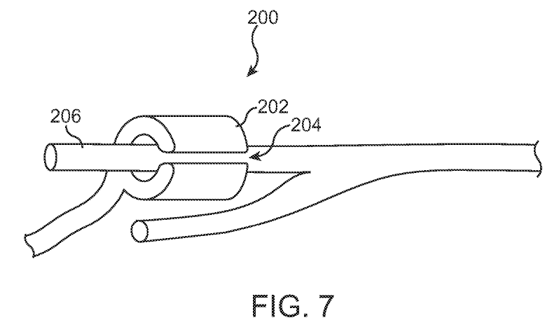

Referring to FIG. 7, another embodiment of an implantable exterior vessel electrostimulation system 200 is depicted comprising a resilient cuff having a body portion 202 and an electrode assembly positioned on a surface of body portion 202. Suitable electrode assemblies and configurations are described above. As depicted in FIG. 4, body portion 202 can comprise a hollow generally cylindrical body defining a gap 204 that permits access into the hollow interior. In one embodiment, body portion 202 can comprise a generally C-shaped cross section. Body portion 202 can be shiftable with respect to a longitudinal axis to allow adjustment of gap 204 from a closed position to an open position. Generally, body portion 202 is biased towards a closed position where gap 204 is slightly smaller than the diameter of the biological vessel structure that system 200 is adapted to fit around. In one embodiment, body portion 202 can be biased such that gap 204, in the closed position, is slightly smaller than the diameter of the carotid sinus artery 206. In these embodiments, body portion 202 can be applied to biological structure 206 by spreading gap 204 and placing body portion 202 around the biological structure. Nerve cuffs having a hollow generally cylindrical body defining a gap are described in U.S. Pat. No. 5,038,781, entitled “Multi-Electrode Neurological Stimulation Apparatus,” which is hereby incorporated by reference herein.

As described above, body portion 202 can be formed from any material suitable for medical device applications including, for example, elastomers, polymers, memory metals and combinations thereof. For example, body portion 202 can be formed from a single material such as silicone rubber, while in other embodiments body portion 102 can be formed by encapsulating a memory metal in a coating selected from the group consisting of polymers, elastomer and blends and copolymers thereof.

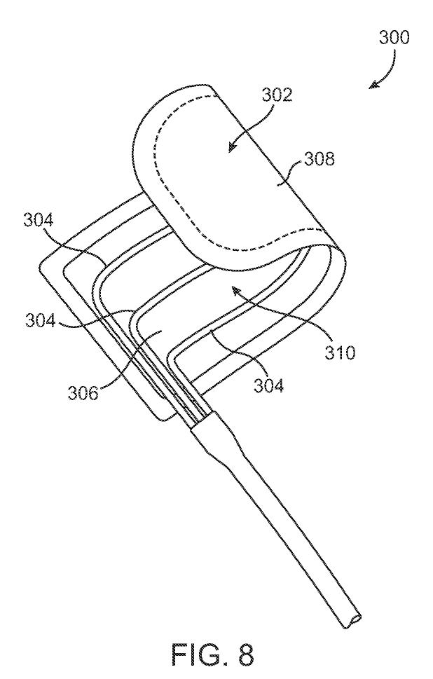

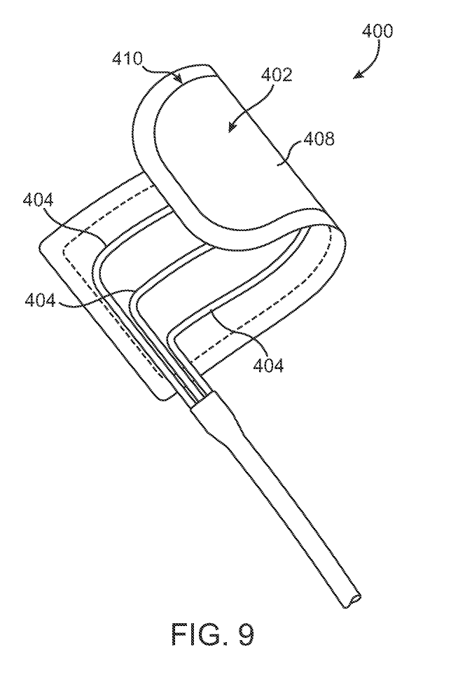

Referring to FIGS. 8 and 9, further embodiments of implantable exterior vessel electrostimulation system comprise an electrode structure disposed on body of a first cuff and a second separate resilient cuff that can be operably coupled to the first cuff to provide a biased, curled shape to the first cuff and the electrodes thereon.

Referring to FIG. 8, an implantable exterior vessel electrostimulation system 300 is depicted comprising a first cuff 302 and an electrode assembly having a plurality of electrodes 304 positioned on a first surface 306 of first cuff 302. First cuff can have first generally planar inner surface 306 and a second generally planar surface 308 opposite first surface 306. A second resilient cuff 310 can be operably coupled or connected to second generally planar surface 308 of first cuff 302 to provide the self-biasing to first cuff 302. Phantom line in FIG. 8 represents the border of first cuff 302 hidden in the view by second cuff 310. Such biasing can enable said first cuff 302 to generally conform to at least a portion of an artery yet substantially enabling normal pulsatile expansion of the artery while maintaining effective artery-electrode interface.

Referring to FIG. 9, implantable exterior vessel electrostimulation system 400 is depicted comprising a first cuff 402 and an electrode assembly having a plurality of electrodes 404 positioned on a surface of first cuff 402. First cuff can have a first generally planar inner surface 406 and second generally planar surface 408 opposite first surface 406. A second resilient cuff 410 can be coupled or connected to second generally planar surface 408 of said first cuff having electrodes thereon. Phantom line in FIG. 9 represents the border of second cuff 410 hidden in the view by first cuff 402. Second cuff 410 can provide self-biasing to first cuff enabling said first cuff 402 to conform to at least a portion of an artery yet substantially enabling normal pulsatile expansion of the artery while maintaining effective artery-electrode interface. In this embodiment, second resilient cuff 408 comprises a frame-like configuration extending around a border of first cuff 402. In this configuration, the biasing force of second cuff 410 is presented at points or positioned distal from the electrodes (i.e., around a perimeter of first cuff 042), and thus distal from the biological features (e.g., baroreceptors) that electrodes 404 are positioned proximate thereto. As a result, second cuff 410 can function to secure device 100 in contact with desired surfaces of a biological structure, while not overly inhibiting pulsatile expansion or interfere with baroreceptor functioning.

In one embodiment, during use of the cuffs of the present disclosure, the body portion of the cuff can be shifted from the biased closed position to an open position. The body portion in the open position can then be positioned proximate a desired surface of a biological vessel structure such as, for example, an artery in the region of the carotid sinus artery. The body portion can then be allowed to return to the biased closed position, which can wrap the body portion of the cuff around the biological vessel structure and can place the electrode assembly in contact with a surface of the biological vessel structure. The position of the cuff can be tested by applying electrical stimulation to the biological vessel structure and monitoring a response such as a baroreflex signal. The above procedure can be repeated until an optimal position for the cuff, and associated electrode assembly, is determined. Once an optimal position for the cuff has been determined, optional fingers can be wrapped around the biological vessel structure and secured to the body portion to provide for additional securing of the cuff to the biological vessel structure.

The embodiments above are intended to be illustrative and not limiting. Additional embodiments are within the claims. Although the present invention has been described with reference to particular embodiments, workers skilled in the art will recognize that changes may be made in form and detail without departing from the spirit and scope of the invention.

What is claimed is:

1. A method of providing stimulation to an artery for purposes of eliciting a baroreflex, the method comprising:
providing a cuff having at least one electrode presented on an inner surface thereof, wherein said cuff is biased to conform to at least a portion of the artery to maintain an intimate artery-electrode interface;
selectively positioning said cuff at a first position on the artery;
enabling said cuff to biasedly conform to at least a portion of the artery to stabilize said cuff proximate said first position; and
activating, deactivating, or otherwise modulating said electrode to provide stimulation to the artery for purposes of eliciting a baroreflex, wherein said cuff comprises resiliency substantially enabling normal pulsatile expansion of the artery while maintaining effective artery-electrode interface.

2. The method of claim 1, further comprising chronically stabilizing said cuff with the artery proximate said first position.

3. The method of claim 2, wherein the chronic stabilization comprises suturing at least a portion of said cuff to the artery.

4. The method of claim 2, wherein the chronic stabilization comprises applying biological glue to at least a portion of said cuff and adhering said biological glue to the artery.

5. The method of claim 2, wherein said inner surface comprises a surface feature and the chronic stabilization comprises enabling arterial tissue growth with said surface feature.

6. The method of claim 1, wherein said resiliency is sufficient to keep said electrode structure in contact with the artery without impeding pulsatile expansion by more than about 80% percent with a pulse pressure of up to about 50 mm Hg.

7. The method of claim 1, wherein said resiliency is sufficient to keep said electrode structure in contact with the artery without impeding pulsatile expansion by more than about 60% percent with a pulse pressure of up to about 50 mm Hg.

8. The method of claim 1, wherein said resiliency is sufficient to keep said electrode structure in contact with the artery without impeding pulsatile expansion by more than about 40% percent with a pulse pressure of up to about 50 mm Hg.

9. The method of claim 1, wherein said resiliency is sufficient to keep said electrode structure in contact with the artery without impeding pulsatile expansion by more than about 20% percent with a pulse pressure of up to about 50 mm Hg.

10. The method of claim 1, further comprising:
effecting said cuff to an open configuration such that movement of said cuff can be effected relative to the artery;
selectively positioning said device at a second position on the artery;
enabling said cuff to conform to at least a portion of the artery proximate said second position; and
activating, deactivating, or otherwise modulating said electrode to provide stimulation to the artery for purposes of eliciting a baroreflex.

11. The method of claim 10, further comprising chronically stabilizing said cuff with artery.

12. The method of claim 10, further comprising comparing the physiologic responses observed at said first and second positions to determine an implant position.

13. The method of claim 12, further comprising effective movement of said device to said implant position and enabling said cuff to biasedly conform to at least a portion of the artery proximate said implant position.

14. The method of claim 13, further comprising activating, deactivating, or otherwise modulating said electrode to provide stimulation to the artery at said implant position for purposes of eliciting a physiologic response.

15. The method of claim 1, wherein said cuff comprises a strap and a buckle, the step of enabling said cuff to biasedly conform to at least a portion of the artery further comprising engaging said strap with said buckle, such that said buckle retains at least a portion of said strap.

16. The method of claim 1, wherein the artery comprises one or more baroreceptors therein, the method further comprising determining said first position comprising determining a location of the one or more baroreceptors and effecting movement of said cuff such that said cuff is proximate said location of said one or more baroreceptors.

17. A cuff selectively positionable on an artery for providing stimulation to the artery for purposes of eliciting a baroreflex, said cuff comprising:
a length and generally opposed first and second edges;
generally opposed inner and outer surfaces; and
an electrode structure presented on said inner surface operable to provide stimulation to an artery,
wherein said cuff is biased to a curled configuration enabling said cuff to conform to at least a portion of the artery to maintain an intimate artery-electrode structure interface for eliciting a baroreflex when said electrode structure is activated, deactivated, or otherwise modulated, and wherein said cuff comprises resiliency enabling normal radial expansion of the artery while maintaining effective artery-electrode interface.

18. The cuff of claim 17, further comprising a chronic stabilization mechanism.

19. The cuff of claim 18, wherein said chronic stabilization mechanism is selectively presented along the first edge.

20. The cuff of claim 18, wherein said chronic stabilization mechanism comprises a surface feature included on said inner surface.

21. The cuff of claim 17, wherein said electrode structure extends transversely with respect to said length from proximate said first edge towards said second edge.

22. The cuff of claim 17, wherein said resiliency is sufficient to keep said electrode structure in contact with the artery without impeding pulsatile expansion by more than about 80% percent with a pulse pressure of up to about 50 mm Hg.

23. The cuff of claim 17, wherein said resiliency is sufficient to keep said electrode structure in contact with the artery without impeding pulsatile expansion by more than about 60% percent with a pulse pressure of up to about 50 mm Hg.

24. The cuff of claim 17, without impeding pulsatile expansion by more than about 40% percent with a pulse pressure of up to about 50 mm Hg.

25. The cuff of claim 17, wherein said resiliency is sufficient to keep said electrode structure in contact with the artery without impeding pulsatile expansion by more than about 20% percent with a pulse pressure of up to about 50 mm Hg.

26. The cuff of claim 17, wherein said electrode structure comprises at least two elongate electrodes.

27. A method of providing medical implants and instruction therefore comprising:
providing a cuff having at least one electrode presented on an inner surface thereof, wherein said cuff is biased to conform to at least a portion of a vascular structure to maintain an intimate vascular structure-electrode interface; and
providing instructions to:
selectively position said cuff at a first position on the vascular structure;
enable said cuff to biasedly conform to at least a portion of the vascular structure proximate said first position; and
activate, deactivate, or otherwise modulate said electrode to provide stimulation to the vascular structure for purposes of eliciting a physiologic response, wherein said cuff comprises resiliency enabling pulsatile expansion of the vascular structure while maintaining effective vascular structure-electrode interface.

28. The method of claim 27, further comprising providing instructions to:
effect said cuff to an open configuration such that movement of said cuff can be effected relative to the vascular structure;
selectively position said device at a second position on the vascular structure;
enable said cuff to conform to at least a portion of the vascular structure proximate said second position; and
activate, deactivate, or otherwise modulate said electrode to provide stimulation to the vascular structure for purposes of eliciting a physiologic response.

29. The method of claim 28, further comprising instructions to:
compare the physiologic responses observed at said first and second positions to determine an implant position.

30. The method of claim 29, further comprising providing instructions to:
effect movement of said device to said implant position and enable said cuff to biasedly conform to at least a portion of the vascular structure proximate said implant position.

31. The method of claim 30, further comprising providing instructions to:
activate, deactivate, or otherwise modulate said electrode to provide stimulation to the vascular structure for purposes of eliciting a physiologic response.

32. The method of claim 27, wherein the vascular structure comprises a carotid artery having one or more baroreceptors therein, the method further comprising providing instructions to:
determine a location of said one or more baroreceptors; and
effect movement of said cuff such that said cuff is proximate said location of said one or more baroreceptors.

33. The method of claim 27, further comprising providing instructions to:
chronically stabilize said cuff with the artery proximate said first position.

34. The method of claim 27, wherein said cuff comprises a strap and a buckle, the method further comprising providing instructions to:
engage said strap with said buckle, such that said buckle retains at least a portion of said strap.

35. A method of providing stimulation to an artery for purposes of eliciting a baroreflex, the method comprising:
providing a first cuff having at least one electrode presented on an inner surface thereof and a second cuff operably coupled to said first cuff, said second cuff biased to conform said first cuff to at least a portion of an artery to maintain an intimate artery-electrode interface;
selectively positioning said first cuff at a first position on the artery;
enabling said second cuff to biasedly conform said first cuff to at least a portion of the artery to stabilize said first cuff proximate said first position; and
activating, deactivating, or otherwise modulating said electrode to provide stimulation to the artery for purposes of eliciting a baroreflex, wherein said first and second cuffs comprise resiliency substantially enabling normal pulsatile expansion of the artery while maintaining effective artery-electrode interface.

36. The method of claim 35, wherein said second cuff is positioned around a border of said first cuff, wherein said biasedly conforming is directed around the border of said first cuff.
